(19) United States
(12) Patent Application Publication
(10) Pub. No.: US 2015/0065863 A1
(43) Pub. Date: Mar. 5, 2015

(54) Positioning Apparatus, Patient Support Apparatus, and Medical Imaging Apparatus

(71) Applicants: Yvonne Candidus, Furth (DE); Daniel Driemel, Oederan (DE); Hubertus Fischer, Bamberg (DE); Wolfgang Kraus, Furth (DE); Thomas Kundner, Buckenhof (DE); Martin Zigann, Brunn (DE); Stephan Zink, Erlangen (DE)

(72) Inventors: Yvonne Candidus, Furth (DE); Daniel Driemel, Oederan (DE); Hubertus Fischer, Bamberg (DE); Wolfgang Kraus, Furth (DE); Thomas Kundner, Buckenhof (DE); Martin Zigann, Brunn (DE); Stephan Zink, Erlangen (DE)

(21) Appl. No.: 14/471,127

(22) Filed: Aug. 28, 2014

(30) Foreign Application Priority Data
Sep. 3, 2013 (DE) 102013217536.1

(51) Int. Cl. A61B 5/055; A61G 13/08

(52) U.S. Cl. CPC A61B 5/0555; A61G 13/08
USPC 600/415; 128/845

(57) ABSTRACT

The embodiments are based on a positioning apparatus designed to provide assistance by supporting and/or positioning a patient on a patient support apparatus. The patient support apparatus includes a bearing unit to hold the patient and a fastening unit. The fastening unit is designed to fasten the positioning apparatus to the patient support apparatus. The bearing unit includes at least two bearing regions, where it is possible to set a position of the first bearing region in relation to a position of the at least one second bearing region.

### CROSS-REFERENCE TO RELATED APPLICATIONS

This application claims the benefit of DE 10 2013 217 536.1, filed on Sep. 3, 2013, which is hereby incorporated by reference in its entirety.

### BACKGROUND

Medical imaging examinations on patients are often quite long, lasting an hour or more. During this time the patient has to remain lying in an examination position on a patient support apparatus, moving as little as possible.

### SUMMARY AND DESCRIPTION

The scope of the present invention is defined solely by the appended claims and is not affected to any degree by the statements within this summary. The present embodiments may obviate one or more of the drawbacks or limitations in the related art.

The object of the present embodiments is to make a patient more comfortable when lying down while maintaining the reproducibility of the patient's horizontal position.

The embodiments are based on a positioning apparatus designed to provide assistance by supporting and/or positioning a patient on a patient support apparatus, with a bearing unit to hold the patient and a fastening unit designed to fasten the positioning apparatus to the patient support apparatus.

It is proposed that the bearing unit has at least two bearing regions, it being possible to set the position of the first bearing region in relation to the position of the at least one second bearing region. This allows the comfort of the patient lying on the patient support apparatus to be improved, in that the positioning apparatus may be used to set the position of the bearing regions. It also allows a setting to be tailored individually to the anatomy of the patient, thereby improving patient comfort. The fact that the bearing regions may be set also provides that the setting is reproducible. The positioning apparatus, in particular the bearing regions of the positioning apparatus, may be designed for the positioning and/or holding of the patient's legs. A patient with raised legs and/or bent legs feels more relaxed and comfortable over a long period lying down and/or a long examination period than when his/her legs are supported and/or positioned in an extended state. To this end, the positioning apparatus may be designed to be positioned on the patient support apparatus, (e.g., in a region for supporting the patient's legs, on the table or the support surface of the table), thereby assisting with the support and/or positioning of the patient, (e.g., the legs of the patient), in a comfortable position, (e.g., in a raised position and/or a bent position).

It is further proposed that at least one of the bearing regions has at least one bearing element with a maximum thickness of 1 cm. In certain embodiments, the bearing element may have a maximum thickness of 5 mm, a maximum thickness of 2.5 mm, or a maximum thickness of maximum 1 mm. The embodiments allow for an essentially smooth transition to be achieved between the at least one bearing element and, for example, a support surface of a table of the patient support apparatus. The at least one bearing element of the positioning apparatus here is supported on the support surface of the table, thereby allowing a high level of comfort to be achieved for the patient lying thereon. The positioning apparatus here may be embodied in such a manner that the at least two bearing regions of the positioning apparatus each have at least one bearing element or that the at least two bearing regions have a common bearing element. The thin configuration of the positioning apparatus, (e.g., the bearing element), also allows the distance between the patient and a high-frequency receive unit for a magnetic resonance examination integrated within the table to be minimized, so that unwanted signal attenuation as a result of the positioning apparatus is prevented.

In a further embodiment, it is proposed that at least one of the bearing regions has at least one bearing element, which includes a flat textile structure. The flat textile structure of the at least one bearing element allows tailoring of the at least one bearing element to a patient, (e.g., to a contour and/or anatomy of a leg region of the patient), to be achieved. This embodiment also allows a thin and light bearing element to be provided to support and/or position the patient. In this context, a flat textile structure refers in particular to a fabric, for example, a material and/or a synthetic fabric, and/or a mesh, and/or a non-woven fabric and/or further flat textile structures that appear expedient to the person skilled in the art.

The at least two bearing regions may also have a common bearing element that has at least one cutout in a peripheral region of the bearing element in a center region. The at least one cutout allows a flattened and, in particular, edgeless transition to be achieved between the first bearing region and the second bearing region in a position where the two bearing regions are at an angle smaller than 180°, thereby allowing a high level of comfort for the patient lying thereon. To this end, the at least one cutout is arranged in a transition region between the first bearing region and the second bearing region. To this end, the common bearing element may have two cutouts arranged on opposing peripheral regions. The cutout(s) may be configured as semi-circular.

In one development, at least one of the bearing regions has at least two strip elements arranged at the side, the at least two strip elements being arranged on different sides of the bearing region and a bearing element being arranged between the at least two strip elements, thereby allowing stability of the positioning apparatus, (e.g., the bearing element of the positioning apparatus), to be achieved. The strip elements here may be formed by profile strip elements and/or a further embodiment that appears expedient to the person skilled in the art.

The bearing unit may have at least one bracing element that is arranged between the two strip elements, thereby allowing stabilizing of a position of and/or a distance between the two strip elements in relation to one another to be achieved, in particular, when the bearing unit is loaded. The at least one bracing element may include a form of cross member, which is arranged between the two strip elements. The bracing element here may be arranged in a center region of the bearing regions in the direction of a longitudinal extension of the positioning apparatus.

It is further proposed that the bracing element has a soft sheathing, thereby allowing a high level of comfort to be achieved for the patient when being positioned and/or supported on the positioning apparatus. To this end, the bracing element may be arranged on at least one of the bearing regions on a side facing away from the bearing surface, so that the supporting and/or positioning of the patient is not obstructed. In this context, a soft sheathing refers, in particular, to a sheathing of the bracing element that may have elastic deformability, so that when the patient is supported, the force of the weight of the patient may be absorbed in the form of an elastic deformation and when the patient leaves the positioning apparatus, the bracing element returns to its original form. For example, the soft sheathing may include a foam material and/or further materials that appear expedient to the person skilled in the art.

It is further proposed that each of the bearing regions has at least one strip element, the strip elements of the different bearing regions being arranged one behind the other on the same side of the bearing unit in the direction of a longitudinal extension of the strip elements. This allows the individual bearing regions of the positioning apparatus to be moved and/or set in relation to one another, thereby allowing the positioning apparatus to be tailored to the anatomy and/or leg position of the patient.

The bearing unit may have at least one rotary bearing unit, with at least two of the strip elements arranged one behind the other being connected by the rotary bearing unit. The rotary bearing unit may include a pivot joint, so that the individual bearing regions of the positioning apparatus may also be moved and/or set in relation to one another, thereby allowing the positioning apparatus to be tailored to the anatomy and/or leg position of the patient. The connection by the rotary bearing unit also provides that the positions of the at least two bearing regions may be set simultaneously, so that when a position of the at least one first bearing region is set, the position of the at least one second bearing region is also fixed at the same time, thereby resulting in a short positioning time when the patient is being prepared in particular for a medical imaging examination.

In a further embodiment, at least two of the strip elements each have an end region at an end facing away from the other strip element in each instance, it being possible to set the distance between the two end regions of the strip elements in order to set a position of the first bearing region in relation to a position of the second bearing region. It is thus possible to set a bearing position of the at least two bearing regions in a structurally simple manner, in that, for example, the two bearing regions together with a support surface of the patient support apparatus enclose a triangular area. The shorter the distance between the end regions of the different strip elements here, the more acute the angle between the bearing regions and a support surface of a table of the patient support apparatus and/or the smaller the angle between the two bearing regions.

If, when the end regions of the different strip elements are at a maximum distance from one another, the different strip elements are at an angle smaller than 180°, it is possible to set a position of the bearing regions in relation to one another economically in a structurally simple manner, (e.g., by a control cable). It is also thus possible to prevent the obstruction, for example, by bracing elements that are arranged on a side of the bearing regions facing away from the bearing surface of the bearing element, of the support and/or positioning of the patient. Because the angle is smaller than 180°, space is available between the bearing regions and a support surface of a patient support apparatus and the bracing elements on the bearing regions may be arranged in this space.

It is further proposed that the bearing unit has a setting unit for setting a position of the first bearing region in relation to a position of the at least one second bearing region, the setting unit including a pneumatic setting unit and/or an electric setting unit and/or a mechanical setting unit and/or a hydraulic setting unit. It is thus possible to set a support position of, in particular, a leg region of the patient, assisting a medical operator, (e.g., a physician, etc.), during an operation to support and/or position a patient on a patient support apparatus, (e.g., on a table of a patient support apparatus). It is also possible to achieve reproducibility of the set support position of, in particular, a leg region of the patient using the setting unit together with the positioning apparatus. The pneumatic setting unit may include, for example, a pneumatic cylinder and/or an at least partially pneumatically driven setting element. The hydraulic setting unit may also include a hydraulic cylinder. The mechanical setting unit may include a control cable and/or further mechanical setting elements that appear expedient to the person skilled in the art. The electric setting unit may include, for example, an electric setting element.

A structurally simple and space-saving setting unit within the positioning apparatus may be provided, if the setting unit includes at least one control cable unit. This provides that the positioning apparatus may be used for a medical imaging examination, which includes a magnetic resonance examination using a magnetic resonance apparatus, as it is possible to prevent unwanted impairment of an applied magnetic field using the mechanical setting unit, (e.g., the control cable unit). The mechanical setting unit here, (e.g., the control cable unit), may be configured as magnetic resonance-compatible.

In a further embodiment, it is proposed that the setting unit has at least one locking element to lock a position of the first bearing region in relation to a position of the second bearing region. A set position of the first bearing region and/or the second setting region may be secured so that an identical examination position is available for the patient (e.g., for different medical imaging examinations). The locking element here may interact with at least one strip element of the positioning apparatus and/or with further units or elements of the positioning apparatus that appear expedient to the person skilled in the art to lock a latching connection and/or a clamping connection.

The locking element may be arranged within the setting unit in such a manner that it may be moved in the direction of a longitudinal extension of the locking element and in a rotation direction about a longitudinal axis of the locking element, thereby allowing simple securing and/or locking of the locking element to be achieved. Provisions may also be made for the locking element to be configured at least partially as a single piece with a setting element and/or a sliding bearing element of the positioning apparatus.

In one development, it is proposed that the fastening unit has at least one sliding bearing element, allowing simple and direct fastening of the positioning apparatus to a patient support apparatus, (e.g., a table of the patient support apparatus), to be achieved. In certain embodiments, the fastening unit may have at least two sliding bearing elements, so that the positioning apparatus may be fastened to the patient support apparatus, (e.g., a table of the patient support apparatus), on both sides and therefore securely. The sliding bearing element may be configured in the same way as a sliding bearing element of the patient support apparatus, (e.g., a table of the patient support apparatus). If, for example, the table has a sliding bearing element that includes a slide rail with an L-shaped cross section, the at least one sliding bearing element of the positioning apparatus is also configured as L-shaped. Also, an L-shaped configuration of the at least one sliding bearing element of the positioning apparatus allows simple securing of the positioning apparatus to the patient support apparatus to be achieved, to prevent the positioning apparatus falling out and/or falling off.

The at least one sliding bearing element is also arranged on one of the end regions of the strip elements, allowing a compact positioning apparatus to be provided. The fastening apparatus may include four sliding bearing elements, one of the sliding bearing elements being arranged in each instance on an end region of one of the strip elements. The sliding bearing elements here are arranged on the end regions of the strip elements 205, 206 of the first bearing region 202, which face away from the second bearing region 203, and on the end regions of the strip elements 205, 206 of the second bearing region 203, which face away from the first bearing region 202.

The embodiments are also based on a patient support apparatus designed to support a patient for a medical imaging examination within a medical imaging apparatus. The apparatus includes a table having a support surface to support the patient, and a positioning apparatus designed, in particular, to provide assistance by supporting and/or positioning a patient on the patient support apparatus, with a bearing unit to hold the patient and a fastening unit that is designed to fasten the positioning apparatus to the patient support apparatus. The bearing unit includes at least two bearing regions, where it is possible to set a position of the first bearing region in relation to a position of the at least one second bearing region. This positioning improves the comfort of a patient lying on the patient support apparatus, in that a position of the bearing regions may be set using the positioning apparatus. Also, the positioning/setting may thus be tailored individually to the anatomy of the patient, thereby improving patient comfort. The fact that the bearing regions may be set also provides that the setting is reproducible.

In a further embodiment of the patient support apparatus, it is proposed that the table has at least one sliding bearing element that is configured in the same way as a sliding bearing element of the positioning apparatus. This allows the positioning apparatus to be fastened in a simple and direct manner to the patient support apparatus, (e.g., the table of the patient support apparatus).

The positioning apparatus may be introduced and/or fastened to the table, if the at least one sliding bearing element of the table includes a slide rail. A slide rail may be arranged on a peripheral region on the table in each instance, the slide rail here being arranged on the table in the direction of a longitudinal extension of the table. The slide rail here may be a groove-type slide rail with an L-shaped cross section.

The embodiments further include a medical imaging apparatus with a patient support apparatus that designed to support a patient for a medical imaging examination within a medical imaging apparatus. The apparatus includes a table having a support surface to support the patient, and a positioning apparatus that is designed to provide assistance by supporting and/or positioning a patient on the patient support apparatus, with a bearing unit to hold the patient and a fastening unit that is designed to fasten the positioning apparatus to the patient support apparatus. The bearing unit includes at least two bearing regions, where it is possible to set a position of the first bearing region in relation to a position of the at least one second bearing region. This positioning improves the comfort of a patient lying on the patient support apparatus, in that a position of the bearing regions may be set and/or tailored individually to the anatomy of the patient using the positioning apparatus. The fact that the bearing regions may be set also provides that the setting is reproducible.

### BRIEF DESCRIPTION OF THE DRAWINGS

FIG. 1 depicts a schematic diagram of an embodiment of a medical imaging apparatus with a patient support apparatus.

FIG. 2 depicts a schematic diagram of an embodiment of a patient support apparatus with a positioning apparatus.

FIG. 3 depicts a schematic diagram of an embodiment of the patient support apparatus with the positioning apparatus in a further support position.

FIG. 4 depicts a rear view of an embodiment of the patient support apparatus with the positioning apparatus in the further support position.

### DETAILED DESCRIPTION

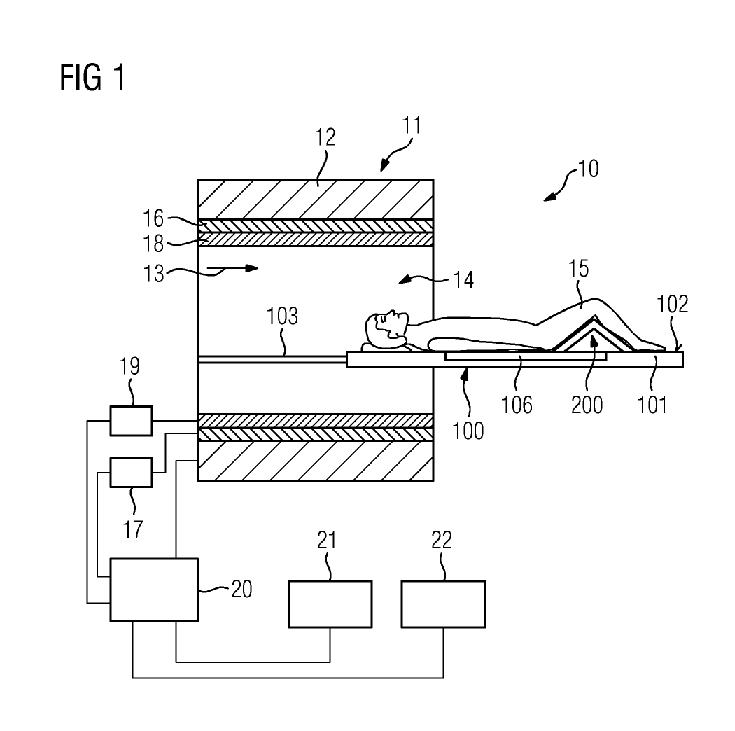

FIG. 1 depicts a schematic diagram of a medical imaging apparatus, which in the present exemplary embodiment is formed by a magnetic resonance apparatus 10. Alternatively, the medical imaging apparatus may also include a computed tomography apparatus, a PET apparatus, a C-arm apparatus, and/or further configurations of a medical imaging apparatus that appear expedient to the person skilled in the art.

The magnetic resonance apparatus 10 includes a magnet unit 11 with a main magnet 12 for generating a powerful and in particular constant main magnetic field 13. The magnetic resonance apparatus 10 also has a patient receiving region 14 for receiving a patient 15. In the present exemplary embodiment, the patient receiving region 14 is configured as cylindrical and is enclosed in a cylindrical manner in a circumferential direction by the magnet unit 11. In certain embodiments, a different configuration of the patient receiving region 14 may be conceivable. The patient 15 may be moved by a patient support apparatus 100 of the magnetic resonance apparatus into the patient receiving region 14.

The magnet unit 11 also has a gradient coil unit 16 for generating magnetic field gradients that are used for spatial encoding during imaging. The gradient coil unit 16 is controlled by a gradient control unit 17 of the magnetic resonance apparatus 10. The magnet unit 11 further includes a high-frequency antenna unit 18 and a high-frequency antenna control unit 19 for exciting a polarization that is established in the main magnetic field 13 generated by the main magnet 12. The high-frequency antenna unit 18 is controlled by the high-frequency antenna control unit 19 and radiates high-frequency magnetic resonance sequences into an examination space that is essentially formed by the patient receiving region 14.

To control the main magnet 12 of the gradient control unit 17 and to control the high-frequency antenna control unit 19, the magnetic resonance apparatus 10 has a control unit 20 formed by a computation unit. The control unit 20 controls the magnetic resonance apparatus 10 centrally, for example, performing a predetermined imaging gradient echo sequence. The control unit 20 also includes an evaluation unit for evaluating image data. Control information, such as imaging parameters, for example, and reconstructed magnetic resonance images may be displayed to an operator on a display unit 21, (e.g., on at least one monitor), of the magnetic resonance apparatus 10. The magnetic resonance apparatus 10 also has an input unit 22, by which information and/or parameters may be input by an operator during a measuring operation.

To support and/or position the patient 15 the patient support apparatus 100 has a table 101 with a support surface 102. The table 101 is supported in a movable manner, so that a patient 15 may be introduced into the patient receiving region 14 of the magnetic resonance apparatus 10 by the table 101. The patient support apparatus 100 also has a base unit 103, the table 101 being supported in such a manner that it may be moved in relation to the base unit 103. To this end, the patient support apparatus 100 has a sliding bearing unit for movable support of the table 101 in relation to the base unit 103. In the present exemplary embodiment, the base unit 103 is integrated within a housing of the magnet unit 11 enclosing the patient receiving region 14.

The patient support apparatus 100 also includes a high-frequency antenna receive unit 106, which is integrated within the table 101 and is designed to detect magnetic resonance signals during a magnetic resonance examination on a patient 15.

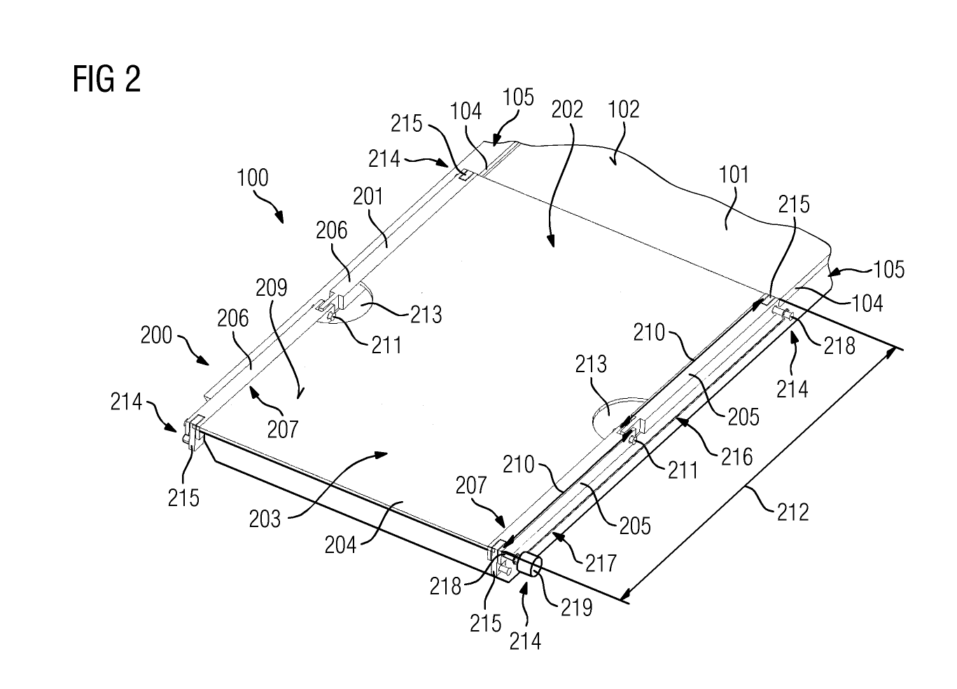

The patient support apparatus 100 further includes a positioning apparatus 200, which is designed to provide assistance by supporting and/or positioning the patient 15 on the patient support apparatus 100. To this end, the positioning apparatus 200 is arranged on the table 101, in particular, on the support surface 102 of the table 101. FIGS. 2 to 4 depict the table 101 of the patient support apparatus 100 in more detail together with the positioning apparatus 200.

The positioning apparatus 200 includes a bearing unit 201, which is designed to hold the patient 15. In the present exemplary embodiment, the bearing unit 201 includes two bearing regions 202, 203 that are designed, in particular, to hold the legs of the patient 15. To this end, the positioning apparatus 200 is arranged in a leg region of the support surface 102 of the table 101 (FIGS. 2 to 4). In principle, the bearing unit 201 of the positioning apparatus 200 may also include more than two bearing regions 202, 203.

The two bearing regions 202, 203 include a common bearing element 204, which includes a flat textile structure in the present instance. The flat textile structure may include a fabric and/or mesh and/or a knitted fabric, etc. In the present exemplary embodiment, the common bearing element includes a fabric formed by a material or a cloth. The common bearing element 204 also may have a maximum thickness of 1 cm, 5 mm, 2.5 mm, or 1 mm, so that there is the smoothest possible transition between the table 101, (e.g., the support surface 102 of the table 101), and the bearing element 204 (FIGS. 2 to 4). The thin configuration of the bearing element 204 also provides that the distance between the patient 15 and the high-frequency antenna receive unit 106 for a magnetic resonance examination integrated within the table 101 may be minimized so that unwanted signal attenuation as a result of the positioning apparatus 200 is prevented.

The bearing unit 201 of the positioning apparatus 200, in particular, the two bearing regions 202, 203, each have two strip elements 205, 206 that are arranged on peripheral regions 207 on opposing sides of the bearing regions 202, 206. In the present exemplary embodiment, the strip elements 205, 206 are formed by profile strips. The bearing element 204 is arranged between the two strip elements 205, 206 of a respective bearing region 202, 203, the bearing element 204 being fastened to the strip elements 205, 206 (FIGS. 2 to 4).

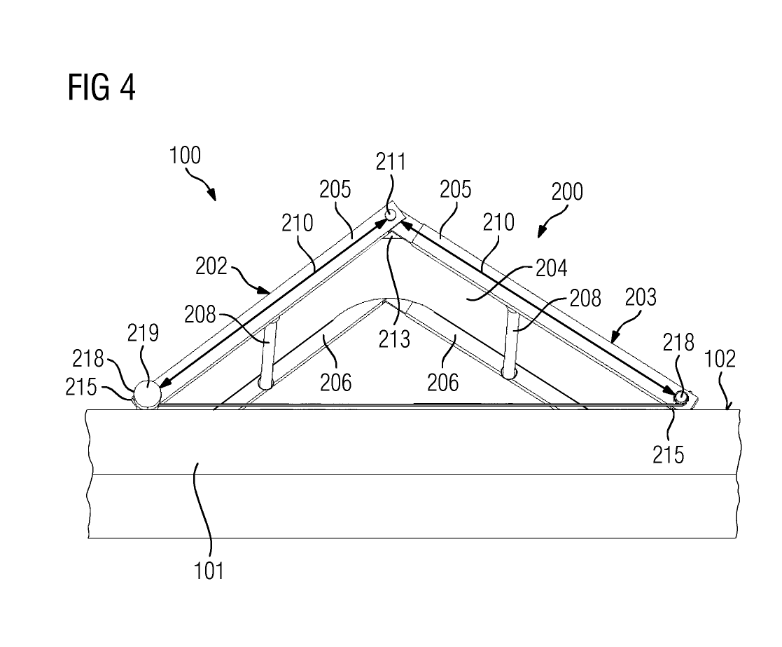

The bearing unit 201 also includes two bracing elements 208, each bearing region 202, 203 here including a bracing element 208 (FIG. 4). The bracing element 208 is arranged between the two strip elements 205, 206 of one of the bearing regions 202, 203 in each instance, so that the distance between the two strip elements 205, 206 remains the same even when the bearing regions 202, 203 are in different positions in relation to one another and/or with different loads, for example because of a patient 15. The bracing elements 208 are arranged in each instance on a side of the bearing element 204 facing away from a support surface 209 of the bearing element 204 between the two strip elements 205, 206 of the bearing regions 202, 203.

The individual bracing elements 208 also have a soft sheathing. The soft sheathing may be a sheathing including a foam material for example and/or further materials that appear expedient to the person skilled in the art. The sheathing of the bracing element 208 may be configured as magnetic resonance-compatible.

To improve the comfort of the patient 15 during a medical imaging examination the two bearing regions 202, 203 are configured so that they may be set in relation to one another. To this end, it is possible to set a position of the first bearing region 202 in relation to a position of the second bearing region 203. A strip element 205, 206 of the first bearing region 202 and a strip element 205, 206 of the second bearing region 203 are arranged on the same side of the bearing unit 201, the two strip elements 205, 206 here being arranged one behind the other in the direction of a longitudinal extension 210 of the strip elements 205, 206. The two strip elements 205, 206 arranged one behind the other are connected by a rotary bearing unit 211 of the bearing unit 201, the bearing unit 201 including two rotary bearing units 211 for this purpose. The rotary bearing units 211 are arranged in each instance between a strip element 205, 206 of the first bearing region 202 and a strip element 205, 206 of the second bearing region 203 and include a pivot joint. One rotary bearing unit 211 in each instance is arranged in an end region of the strip element 205, 206 of the first bearing region 202 that faces the second bearing region 203, and in an end region of the strip element 205, 206 of the second bearing region 203 that faces the first bearing region 202 (FIGS. 2 to 4).

The bearing element 204 has two cutouts 213 in a center region in the direction of a longitudinal extension 212 of the bearing unit 201. In the present exemplary embodiment, the two cutouts 213 are configured as semicircular and are arranged in a peripheral region 207 of the bearing element 204. The cutouts 213 here are arranged in a region of the bearing unit 201, in which the rotary bearing units 211 are arranged.

The positioning apparatus 200 also has a fastening unit 214 that is designed to fasten the positioning apparatus 200 to the patient support apparatus 100. The fastening unit 214 has at least one sliding bearing element 215 to support the positioning apparatus 200 in an at least partially movable manner on the table 101, (e.g., on the support surface 102 of the table 102). In the present exemplary embodiment, the fastening unit 214 has four sliding bearing elements 215 that are arranged on one of the strip elements 205, 206 in each instance. The sliding bearing elements 215 here are arranged on an end region of the strip elements 205, 206 in each instance, the end region of the strip elements 205, 206 being arranged on the strip elements 205, 206 in each instance facing away from the end regions of the strip elements 205, 206 including the rotary bearing unit 211 in the direction of the longitudinal extension 210 of the strip elements 205, 206.

The positioning apparatus 200 is introduced into sliding bearing elements 104 of the table 101 by the sliding bearing elements 215, the sliding bearing elements 215 of the positioning apparatus 200 being configured in the same way as the sliding bearing elements 104 of the table 101. In the present instance, the sliding bearing elements 104 of the table 101 are formed by two slide rails, which are configured in the manner of grooves with an L-shaped cross section. The slide rails extend in the direction of a longitudinal extension of the table 101 and are also arranged in a peripheral region 105 of the support surface 102 of the table 101 in each instance. The sliding bearing elements 215 of the positioning apparatus 200 also include L-shaped webs, allowing advantageous sliding and also securing by the fastening unit 214 to prevent the positioning apparatus 200 falling out (FIG. 2).

The sliding bearing elements 215 on the same end region of the bearing unit 201 are connected by an axle. The individual sliding bearing elements 215 are also supported by a rotary bearing unit in each instance on the strip elements 205, 206, so that it is possible to set the inclination of the individual bearing regions 202, 203 when the positioning apparatus 200 is supported on the table 101. The fastening unit 214 also has a fixing element that may be arranged on one of the sliding bearing elements 215 and is used to fix a position of the positioning apparatus 200 in relation to the patient support apparatus 100, (e.g., in relation to the table 101 of the patient support apparatus 100).

The rotary bearing units 211 and the sliding bearing elements 215 may be used to set a position of the first bearing region 202 in relation to a position of the second bearing region 203 and the positions of the first and second bearing regions 202, 203 in relation to the table 101 of the patient support apparatus 100. To this end, the rotary bearing units 211 and the sliding bearing elements 215 are used to set an angle between the two bearing regions 202, 203. The smaller the angle formed by the two bearing regions 202, 203, the smaller the distance between the end regions of the strip elements 205, 206 supported on the table 101 by the sliding bearing elements 215. Also, a function of the distance between the end regions of the strip elements 205, 206 supported on the table 101 by the sliding bearing elements 215 is an angle formed by the support surface 102 of the table 101 and the support surface 209 of the bearing regions 202, 203. The smaller the distance between the end regions of the strip elements 205, 206 supported on the table 101 by the sliding bearing elements 215, the smaller also the angle between the support surface 102 of the table 101 and the support surface 209 of the bearing regions 202, 203.

When the positioning apparatus 200 is positioned in such a manner that it helps the legs of the patient 15 to be bent and/or raised, the two bearing regions 202, 203 form an angle which may be smaller than 170°. In this position, the cutouts in the bearing element 204 provide that the transition between the first bearing region 202 and the second bearing region 203 is flattened and edgeless, so the patient may be supported and/or positioned in an agreeable and/or comfortable manner.

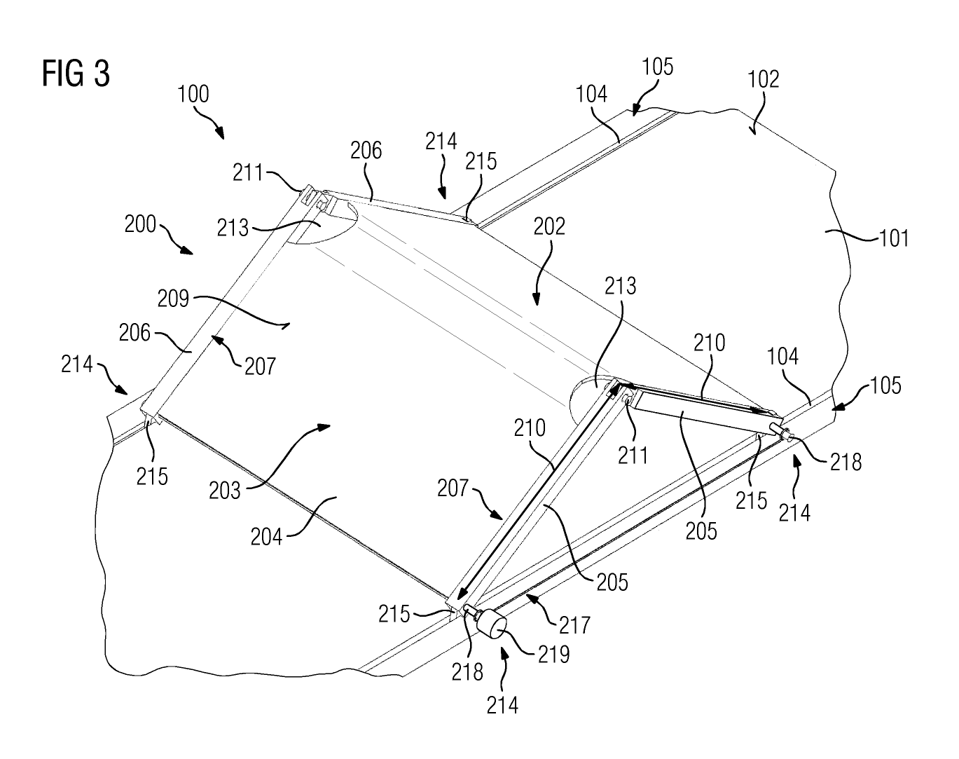

To set the position of the first bearing region 202 in relation to a position of the second bearing region 203, the bearing unit 201 also has a setting unit 216. In the present exemplary embodiment, the setting unit 216 includes a mechanical setting unit 216 with a control cable unit 217 (FIG. 2). To this end, a cylindrical setting element 218 is arranged in each instance on the end regions of the strip elements 205, 206 of the first bearing region 202 facing away from the second bearing region 203 and on the end regions of the strip elements 205, 206 of the second bearing region 203 facing away from the first bearing region 202, the setting element 218 extending away on a side of the strip elements 205, 206 facing away from the bearing element 204. One of the cylindrical setting elements 218 on each side of the positioning apparatus 200 is also supported in such a manner that it may be rotated on the strip elements 205, 206. The two cylindrical setting elements 218 are connected by a cable. In certain embodiments, it is possible for the cable to be rolled up at least partially onto the setting element 218 by rotating the rotatably supported cylindrical setting element 218, thereby allowing a distance to be set between the two cylindrical setting elements 218, and thus also between the two end regions of the strip elements 205, 206 (FIGS. 3 and 4).

In an alternative embodiment, the setting unit 216 may include a pneumatic setting unit 216 and/or an electric setting unit 216 and/or a hydraulic setting unit 216 and/or an alternative embodiment of a mechanical setting unit 216. The pneumatic setting unit 216 may have for example a pneumatic cylinder and/or a pneumatically operated setting element, (e.g., a setting arm). The hydraulic setting unit 216 may include for example a hydraulic cylinder. The electric setting unit 216 may include, for example, an electrically operated setting element. It is also conceivable in an alternative embodiment of the mechanical setting unit 216 for the setting unit to have two control cable units 217 that are arranged only on different sides of the positioning apparatus 200. A pneumatic setting unit and/or an electric setting unit and/or a hydraulic setting unit may be arranged in a region of the rotary bearing unit 211 and/or on the strip elements 205, 206.

To set a position of the first bearing region 202 in relation to a position of the second bearing region 203 on the table 101, the control cable units 217 are used to set a distance between the end regions of the strip elements 205, 206 of the first bearing region 202 facing away from the second bearing region 203 and the end regions of the strip elements 205, 206 of the second bearing region 203 facing away from the first bearing region 202. In a position where there is a maximum distance between the end regions of the strip elements 205, 206, the first bearing region 202 is at an angle smaller than 180° to the second bearing region 203, the two bearing regions 202, 203 potentially being at an angle of approx. 170°, so that the bracing elements 208 do not obstruct the support and/or positioning of the patient 15.

When the end regions of the strip elements 205, 206 are in a position in which the end regions are at a maximum distance from one another, the distance between the end regions of the strip elements 205, 206 may be shortened by rotating one of the cylindrical setting elements 218 of the setting unit 216.

When the end regions of the strip elements 205, 206 are in a position where the distance between the end regions does not correspond to the maximum distance, the distance between the end regions of the strip elements 205, 206 may be both shortened and increased by rotating one of the cylindrical setting elements 218 of the setting unit 216, depending on the rotation direction.

To secure and/or lock a set position of the first bearing region 202 in relation to a position of the second bearing region 203, the setting unit 216 has a locking element 219. The locking element 219 is arranged on the rotatably supported, cylindrical setting element 218 and is configured at least partially as a single piece with this. The locking element 219 is thus supported in such a manner that it may be rotated in a rotation direction about a longitudinal axis of the locking element 219, and therefore also about a longitudinal axis of the cylindrically configured setting element 218. The locking element 219 is also supported in such a manner that it may be moved in the direction of its longitudinal extension in relation to the strip element 205, 206.

The locking element 219 has, for example, a square cross sectional area for securing and/or locking purposes, so that when the locking element 219 is rotated, it becomes jammed against the strip element 205, 206. Further embodiments of the locking element 219 are conceivable, as long as a clamping connection and/or a latching connection results between the locking element 219 and the strip elements 205, 206.

So that the positioning apparatus 200 may be used together with the magnetic resonance apparatus 10, the positioning apparatus 200 is configured as magnetic resonance compatible.

The illustrated patient support apparatus 100 and the illustrated magnetic resonance apparatus 10 may include further components, which patient support apparatuses and magnetic resonance apparatuses may feature. The mode of operation of a magnetic resonance apparatus and patient support apparatus is also known to the person skilled in the art so there is no need for a detailed description of the components here.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A positioning apparatus for supporting, positioning, or supporting and positioning a patient on a patient support apparatus, the positioning apparatus comprising;
a bearing unit configured to hold the patient; and
a fastening unit configured to fasten the positioning apparatus to the patient support apparatus,
wherein the bearing unit comprises a first bearing region and a second bearing region, the first bearing region configured to be positioned in relation to the second bearing region.

2. The positioning apparatus as claimed in claim 1, wherein the first bearing region or the second bearing region comprises at least one bearing element with a maximum thickness of 1 cm.

3. The positioning apparatus as claimed in claim 1, wherein the first bearing region or the second bearing region comprises at least one bearing element, the at least one bearing element comprising a flat textile structure.

4. The positioning apparatus as claimed in claim 1, wherein the first bearing region and the second bearing region comprise a common bearing element, the bearing element comprising at least one cutout in a peripheral region of the bearing element in a center region of the peripheral region.

5. The positioning apparatus as claimed in claim 1, wherein the first bearing region or the second bearing region comprises at least two strip elements arranged at the side of the bearing unit, the at least two strip elements being arranged on different sides of the first bearing region or the second bearing region, and
wherein a bearing element is arranged between the at least two strip elements.

6. The positioning apparatus as claimed in claim 5, wherein the bearing unit comprises at least one bracing element, the at least one bracing element arranged between the two strip elements.

7. The positioning apparatus as claimed in claim 6, wherein the bracing element comprises a soft sheathing.

8. The positioning apparatus as claimed in claim 1, wherein each bearing region of the first and second bearing regions comprises at least one strip element, the strip elements of the first and second bearing regions being arranged one behind the other on a same side of the bearing unit in a direction of a longitudinal extension of the strip elements.

9. The positioning apparatus as claimed in claim 8, wherein the bearing unit comprises at least one rotary bearing unit, wherein at least two of the strip elements arranged one behind the other are connected by the rotary bearing unit.

10. The positioning apparatus as claimed in claim 8, wherein at least two of the strip elements each comprise an end region at an end facing away from the other strip element,
wherein a distance between the end regions of the strip elements is configurable to set a position of the first bearing region in relation to a position of the second bearing region.

11. The positioning apparatus as claimed in claim 10, wherein, when the end regions of the strip elements are at a maximum distance from one another, the different strip elements are at an angle less than 180°.

12. The positioning apparatus as claimed in claim 10, wherein the fastening unit comprises at least one sliding bearing element, and wherein the at least one sliding bearing element is arranged on one of the end regions of the strip elements.

13. The positioning apparatus as claimed in claim 1, wherein the bearing unit comprises a setting unit configured to set a position of the first bearing region in relation to a position of the second bearing region, the setting unit comprising a pneumatic setting unit, an electric setting unit, a mechanical setting unit, a hydraulic setting unit, or a combination thereof.

14. The positioning apparatus as claimed in claim 13, wherein the setting unit comprises at least one control cable unit.

15. The positioning apparatus as claimed in claim 13, wherein the setting unit comprises at least one locking element configured to lock a position of the first bearing region in relation to a position of the second bearing region.

16. The positioning apparatus as claimed in claim 15, wherein the locking element is configured to be moved in a direction of a longitudinal extension of the locking element and in a rotation direction about a longitudinal axis of the locking element.

17. The positioning apparatus as claimed in claim 1, wherein the fastening unit comprises at least one sliding bearing element.

18. A patient support apparatus for supporting a patient for a medical imaging examination within a medical imaging apparatus, the patient support apparatus comprising:
a table comprising a support surface configured to support the patient; and
a positioning apparatus comprising (1) a bearing unit configured to hold the patient; and (2) a fastening unit configured to fasten the positioning apparatus to the patient support apparatus, wherein the bearing unit comprises a first bearing region and a second bearing region, the first bearing region configured to be positioned in relation to the second bearing region.

19. The patient support apparatus as claimed in claim 18, wherein the fastening unit of the positioning apparatus comprises at least one sliding bearing element, and
wherein the table comprises at least one sliding bearing element configured in a same way as the at least one sliding bearing element of the positioning apparatus.

20. The patient support apparatus as claimed in claim 19, wherein the at least one sliding bearing element of the table comprises a slide rail.

21. A medical imaging apparatus comprising:
a magnetic resonance apparatus;
a patient support apparatus configured to support a patient for a medical imaging examination at least partially within the magnetic resonance apparatus, the patient support apparatus comprising a table having a support surface configured to support the patient; and
a positioning apparatus comprising (1) a bearing unit configured to hold the patient; and (2) a fastening unit configured to fasten the positioning apparatus to the patient support apparatus, wherein the bearing unit comprises a first bearing region and a second bearing region, the first bearing region configured to be positioned in relation to the second bearing region.
